(19) United States
(12) Patent Application Publication
(10) Pub. No.: US 2026/0035670 A1
(43) Pub. Date: Feb. 5, 2026

(54) IN VITRO CONSTRUCT USEFUL FOR DRUG TOXICITY SCREENING

(71) Applicant: Wake Forest University Health Sciences, Winston-Salem, NC (US)

(72) Inventor: Yuanyuan ZHANG, Winston-Salem, NC (US)

(21) Appl. No.: 19/100,739

(22) PCT Filed: Aug. 4, 2023
(86) PCT No.: PCT/US2023/071641

§ 371 Date: Feb. 3, 2025

Related U.S. Application Data
(60) Provisional application No. 63/370,418, filed on Aug. 4, 2022.

(51) Int. Cl. C12N 5/071; G01N 33/50

(52) U.S. Cl. CPC C12N 5/0684; G01N 33/5014; G01N 33/5073; C12N 2502/1157; C12N 2502/1352; C12N 2502/28; C12N 2503/02; C12N 2513/00; C12N 2533/50

(57) ABSTRACT

An in vitro construct useful for toxicity testing is provided, comprising: a three-dimensional (3D) scaffold comprising silk fibroin and having a crosslinked porous matrix; and stem cells adherent to the 3D scaffold. In some embodiments, the stem cells adherent to the 3D scaffold maintain stable mitochondrial DNA in long term culture. In some embodiments, the stem cells are urine stem cells.

### REFERENCE TO RELATED APPLICATIONS

This application claims the benefit of U.S. Provisional Patent Application No. 63/370,418, filed Aug. 4, 2022, the contents of which are incorporated by reference herein.

### STATEMENT OF GOVERNMENT SUPPORT

This invention was made with government support under Contract Number R03 AI165170 awarded by the National Institutes of Health. The U.S government has certain rights in the invention.

### BACKGROUND

Antiretroviral treatment (ART) keeps HIV under control, protecting patients' immune systems and allowing them to lead healthier, and longer lives. However, late mitochondrial toxicity (MtT) is a major adverse effect of long-term ART. Long-term use of nucleoside analogues, nucleoside/nucleotide reverse transcriptase inhibitors (NRTIs) and integrase strand transfer inhibitors (INSTIs) cause serious MtT. NRTIs often induce chronic MtT that leads to cardiomyopathy, myopathy, peripheral neuropathy, nephropathy and hepatic steatosis with lactic acidosis, which can be life-threatening. See, e.g., Papatheodoridis et al. The American journal of gastroenterology 2002, 97:1618-28; Carr et al. Lancet 2000, 356:1423-30; Brinkman et al. Curr Opin Infect Dis 2000, 13:5-11; Maagaard et al. Journal of Antimicrobial Chemotherapy 2009, 64:901-9; Rana et al. Chemical Research in Toxicology 2019, 32:156-67; Arnaudo et al. Lancet 1991, 337:508-10; Dalakas et al. N Engl J Med 1990, 322:1098-105; Dalakas et al. Lab Invest 2001, 81:1537-44; Moore et al. AIDS 2000, 14:273-8; Cote et al. Antivir Ther 2006, 11:79-86; Saumoy et al. AIDS 2004, 18:1741-2; Samuels et al. AIDS 2017, 31:1297-301; Brinkman et al. Aids 1998, 12:1735-44; Moyle. Clin Ther 2000, 22:911-36; discussion 898.

Though the older antiretroviral drugs most associated with MtT are now rarely used, newer agents, including INSTIs, have the potential to cause short-term and long-term adverse events, such as weight gain, cardiovascular effects, immune dysfunction, insomnia, and neuropsychiatric disorders, and the underlying mechanism of these adverse effects of INSTIs could be associated with MtT. See, e.g., Eckard et al. Curr Opin Infect Dis 2020, 33:10-9.

A recent study reported that the INSTI dolutegravir (DTG) induces mitochondrial dysfunction in microglia cells, which are the resident brain phagocytes (George et al. Front Immunol 2021, 12:639378). INSTIs (i.e. DTG and raltegravir [RAL]) display pro-adipogenic and pro-lipogenic effects, and promote oxidative stress, mitochondrial dysfunction, and insulin resistance (Gorwood et al. Clinical Infectious Diseases 2020, 71:e549-e60). Furthermore, elvitegravir (EVG) and DTG are associated with underlying MtT, as evidenced by increased levels of mitochondrial ROS and mitochondrial mass, resulting in overall decreased cellular function in CD4+ T cells (Korencak et al. JCI Insight 2019, 4). Other classes of drugs, such as anticancer drugs and antibiotics, are also associated with drug-induced late toxicities including MtT and nephrotoxicity. See, e.g., Will et al. Expert. Opin. Drug Metab. Toxicol. 2014, 10, 1061-1067; Varga et al. American Journal of Physiology-Heart and Circulatory Physiology 2015, 309:H1453-H67; Kamalian et al. Toxicol In Vitro 2018, 53:136-47; Rana et al. Chemical Research in Toxicology 2019, 32:156-67. In addition, drug-induced MtT has been associated with organ toxicity in several solid organs, including the liver, kidney, heart, central nervous system, and skeletal muscle.

Assessment of MtT is vital to minimize the toxicity sequelae to patients and to flag toxic drugs early in development. Despite often being widely used, existing in vitro two-dimensional (2D) culture models of cell lines are not suitable for testing chronic mitotoxicity. Three-dimensional (3D) cultures would be better to assess toxicity (Edmondson et al. Assay Drug Dev Technol 2014, 12:207-18). However, no suitable long-term 3D culture (>4 weeks) assays of primary human cells exist for MtT assessment.

As one of the 3D cell culture models, spheroids have been commonly used in new drug development to closely mimic the main structural and functional features of human solid tissues (Nunes et al. Biotechnol Bioeng 2019, 116:206-26). Human primary hepatocytes remain in 3D spheroids for long-term cultures (3-5 weeks) (Bell et al. Scientific reports 2016, 6:25187; Klaas et al. Scientific reports 2021, 11:20165). However, 3D spheroid systems cannot provide sufficient large-scale cells (such as half million cells/well), because optimal sizes of spheroids require less than 350 μm with cell concentration ranging from 2,000 to 8,000 cells/spheroid to reduce necrosis at the center of 3D spheroids (Guo et al. ACS Biomater Sci Eng 2020, 6:6701-9). Creating large numbers of human primary cells in 3D spheroid cultures is time-consuming, labor-intensive, and expensive (Lee et al. Scientific reports 2019, 9:13976). In addition, the maximal time frame for testing drugs is four weeks in most 3D spheroids with human primary cells (Ding et al. Pharmaceutics 2022, 14:1042).

Therefore, an improved and cost-effective long-term 3D culture method with high cell numbers and good biological stability of primary human cells is needed for testing drug toxicity such as chronic ART-MtT.

### SUMMARY

Provided herein according to some embodiments is an in vitro construct useful for toxicity testing, comprising: a three-dimensional (3D) scaffold comprising silk fibroin and having a crosslinked porous matrix; and stem cells adherent to the 3D scaffold. In some embodiments, the stem cells adherent to the 3D scaffold maintain stable mitochondrial DNA for at least 6 weeks in culture.

In some embodiments, the stem cells are urine stem cells (e.g., USC with telomerase activity).

In some embodiments, the stem cells are autologous to a patient in need of, or a candidate for, long term treatment with a drug.

In some embodiments, the 3D scaffold comprises electrospun silk fibroin. In some embodiments, the electrospun silk fibroin comprises mixed-sized fibers from 1 to 30 micrometers in diameter.

In some embodiments, the 3D scaffold has pores with sizes of from 40 to 80 micrometers.

In some embodiments, the 3D scaffold has a porosity of 80% or greater.

In some embodiments, the in vitro construct is grown as a dynamic culture (e.g., at about 40 RPM on an orbital shaker).

In some embodiments, the construct further comprises macrophages, endothelial cells and/or stromal cells (e.g., human primary macrophages, endothelial cells, stromal cells, and USC provided at a ratio of 1:1:1:7, respectively).

Also provided is a method of making the in vitro construct as taught herein comprising: providing a 3D scaffold comprising silk fibroin and having a crosslinked porous matrix; seeding the 3D scaffold with the stem cells (e.g., USC); allowing the stem cells to adhere to the 3D scaffold; and growing the stem cells on the 3D construct for a time of from 2 or 4 weeks, to 6, 8 or 10 weeks, to thereby make the in vitro construct.

In some embodiments, the providing step is carried out by electrospinning a composition comprising the silk fibroin and then crosslinking the silk fibroin.

Further provided is a method of performing toxicity testing, comprising: providing the in vitro construct as taught herein; contacting the construct with a substance of interest (e.g., a drug) for a period of from 2 to 6 weeks; and detecting a biological response of the stem cells, wherein said biological response indicates toxicity of the substance of interest.

In some embodiments, the biological response of the stem cells indicates cytotoxicity or mitochondrial toxicity in an organ or tissue. In some embodiments, the organ or tissue comprises liver, heart, brain/periphery nerve, skeletal muscle, blood cells, or kidneys (i.e., hepatotoxicity, cardiotoxicity, neurotoxicity, myotoxicity, hematotoxicity or nephrotoxicity, respectively).

In some embodiments, the drug is an antiretroviral therapy (ART) drug. In some embodiments, the drug is selected from the group consisting of: anti-diabetic drugs (such as thiazolidinediones, fibrates, biguanides), cholesterol lowering drugs (such as statins), anti-depressants (such as SARIs), pain medications (such as NSAIDs), antibiotics (such as fluroquinolones, macrolide), and anti-cancer drugs (kinase inhibitors and anthracyclines).

### BRIEF DESCRIPTION OF THE DRAWINGS

FIG. 1. Morphology and viability of USC during long-term culture. Panel A) Morphology of urine stem cells (USC) in three culture conditions with time. USC at passage 4 (p4) grown on silk fibers of small size SFM (s-SFM, 4 mm at diameter and 0.2 mm in thickness), and 3D spheroids remained stable in size in ultra-low attachment 96-well plates. USC reached over-confluent status with time in 2D culture, when USC were initially seeded at 4×10³ at culture conditions (3D sSFM at Ø4 mm×0.2 mm, spheroid and 2D culture in 96-well plate) at different time points, under phase contrast microscope. Panel B) Cell proliferation of USC in culture (initial seeding cell number 4×10³) at day 1, 3, 7 and week 2, 4, 6, and 8, assessed by CCK-8 test. *p<0.05. Panel C) Cell viability of USC in 3D cultures at different time points. In 3D SFM, most USC (95%) survived up to 8 weeks although there was about 20% of cell density decrease at 8 weeks, compared to cultures at 6 weeks. In 3D spheroids, most cells appeared healthy at week 4 but the number of dead cells increased at week 6. The size of USC spheroids presented slightly larger in the first week, remained stable at week 2 and 4, and decreased with time starting at week 6. It formed necrosis at the center of the spheroids at week 8 (assessed by live/dead kit).

FIG. 2. Mitochondrial mass and DNA content (mtDNA content) in 3D cultures. There was no statistical difference in mtDNA content per cell between 3D spheroids and 3D USC-SFM at week 6 (p>0.05). The levels of mtDNA content in 3D of USC-SFM are similar between week 2 and 6, assessed by q-PCR. (4×10³ cells/well in ULA 96-well plate, n=3/sample, *p<0.05).

FIG. 3. Cell concentration in 3D USC-SFM during long-term culture. Number of USC at different cell concentrations at p4 seeded on SFM (O at 4 mm×0.2 mm/well in 96-well plates, n=3) for 8 weeks was assessed by CCK-8, indicating that the maximal number of cells that can be loaded in SFM in a 96-well plate is 1×10⁵ cells/1.3 mm³ SFM/well. When more cells (e.g., 10⁶ cells/25.2 mm³ SFM/well) were loaded into large size SFM, it retained only 1×10⁵ cells up to 8 weeks.

FIG. 4. Significant increases in superoxide dismutase 2 (SOD2) expression in spheroids, but not in 3D USC-SFM. Panel A) Protein levels of mitochondrial SOD2 significantly increased in 3D USC spheroids, compared to that in 3D USC-SFM and 2D culture at weeks 2 and 6, as assessed by Western blot. Panel B) The semi-quantification analysis for SOD2 in cell lysates shown for (A) The intensity of the bands in each cell culture model was calculated relative to β-Actin housekeeping protein. Data (n=3 samples) were expressed as means±SD.

FIG. 5. Senescence-related gene expression of USC in 3D cultures. Quantitative PCR analysis showed the mRNA levels (RB and P16, p21) of USC in 2D culture, 3D spheroids and 3D SFM from three individuals at day 3, week 2 and week 6, respectively. The results were expressed as mean±SD of three independent experiments. Asterisks indicate significant differences (*p<0.05, **p<0.01). Senescence-related genes in 3D USC-SFM were significantly lower compared to those in 3D USC spheroids at week 6.

FIG. 6. Mitochondrial DNA content and mass significantly decrease with increased ddC doses in 3D culture of USC over time. Changes in mtDNA content of USC treated with Zalcitabine (ddC, a nucleoside reverse transcriptase inhibitor). Levels of mtDNA content significantly decreased in 3D spheroid of USC and 3D USC-SFM 2 and 6 weeks after being treated with ddC at three different doses (0.2, 2, or 20 μM), compared to cells treated with DMSO standardized as 100%. ddC at 2 or 20 μM significantly inhibited mtDNA content compared to those treated 0.1 μM ddC. Decreased mtDNA content displayed a dose-dependent manner in 3D spheroids of USC; decreased mtDNA content showed in a time-depended manner in 3D USC-SFM, which was assessed by q-PCR. Fold change of relative mtDNA content was compared to control (0.1% DMSO) n=3 samples) after the values were normalized to nuclear DNA.

FIG. 7. Dysfunctional electron transport chain (ETC) complexes in 3D cultures of USC 6 weeks after ddC treatments. Panel A) Cell viability of 3D USC-SFM treated with ddC over time. USC at p4 seeded on 3D SFM after treatment with ddC (0.1, 2, 10 μM), assessed by CCK-8. Panel B) The semi-quantification analysis showed that activity of complex II-IV significantly decreased in USC-SFM treated with ddC at middle and high doses, compared to those in low dose at week 6 (p<0.5 or p<0.01); Panel C) Curiously, there was a decrease in expression of ETC complex I-IV in USC-SFM and 3D organoids treated with ddC at middle and high doses at wk 6. In addition, USC-SFM seems more sensitive in detecting complex III and IV, compared to 3D organoids of USC.

### DETAILED DESCRIPTION

The present invention is now described more fully hereinafter with reference to the accompanying drawings, in which embodiments of the invention are shown. This invention may, however, be embodied in many different forms and should not be construed as limited to the embodiments set forth herein; rather, these embodiments are provided so that the disclosure will be thorough and complete and will fully convey the scope of the invention to those skilled in the art.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity. All publications, patent applications, patents and other references mentioned herein are incorporated by reference in their entirety. In case of a conflict in terminology, the present specification is controlling.

As used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

### I. Cells

“Cells” used in the present invention are, in general, animal cells, particularly mammalian and primate cells, examples of which include, but are not limited to, human, dog, cat, rabbit, monkey, chimpanzee, cow, pig, or goat. In some embodiments, the cells are primary cells. In some embodiments, the cells are autologous cells from patients (human or other animal) who are taking or considering taking a drug for which drug toxicity testing is to be performed.

“Primary” as used herein and in the context of a primary cell or primary stem cell refers to a cell that has not been transformed or immortalized. Such primary cells can be cultured, sub-cultured, or passaged a limited number of times (e.g., 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, tissue, or bodily fluid, optionally sorted. In some embodiments, cells are utilized directly without culturing or sub-culturing. In some embodiments, cells are used after passaging (e.g., passaged from 2-6 times, or from 3-5 times).

In some embodiments, the cells are stem cells, including but not limited to mesenchymal stem cells, adipose derived stem cells, kidney stem cells, and urine stem cells (USC). In some preferred embodiments, the cells are urine stem cells.

“Urine stem cells” or “USC” are renal stem cells that may be found in, and collected and/or isolated from, urine, which cells possess both pluripotency and proliferative potential. A USC is “pluripotent” in that it is capable of giving rise to various cell types within one or more lineages. For example, USC according to some embodiments possess the potential to differentiate into one or more of the following: bladder urothelial, smooth muscle, endothelium, interstitial cells, and even bone, muscle, epithelial cells and other types of cells and tissues (e.g., fat, cartilage, nerve). USC are further described in U.S. Pat. Nos. 9,700,581, 10,398,804, and 11,135,248 to Zhang et al., which are incorporated by reference herein. In some embodiments, USC are positive for telomerase activity (USC^{TA+}). See Shi et al., Front. Cell and Dev. Bio. 10:890574 (May 2022).

Urine stem cells according to some embodiments of the present invention can be identified, selected, and/or isolated based on one or more “markers.” Such markers include specific gene expression, antigenic molecules found on the surface of such cells, etc. In particular embodiments, urine stem cells are selected and isolated based upon the expression of at least one specific maker. In some embodiments, USC have one or more of the following markers such as CD117 (C-kit), SSEA-4, CD105, CD73, CD90, CD133, and CD44, and do not have an appreciable amount of one or more of the following markers: CD31, CD34, and CD45. Accordingly, certain embodiments embrace selecting and isolating urine stem cells which express one or more of CD117, SSEA-4, CD105, CD73, CD90, CD133, and CD44 and/or lack expression of one or more of CD31, CD34, and CD45. For example, in some embodiments a urine stem cell of the present invention is identified, selected, and/or isolated based on the expression of CD117. Urine stem cells according to some embodiments also express MSC/pericyte markers such as CD146 (MCAM), NG2 (a related antigen), and/or PDGF-Receptorβ (PDGF-Rβ). Marker expression may be probed by methods known in the art, e.g., western blot, RT-PCR, immunofluorescence, FACS, etc. In some embodiments, USC are positive for a marker selected from: CD133, SSEA-A, CD90, CD73, CD105, pericyte CD146 (MCAM), NG2, PDGF-Receptorβ (PDGF-Rβ), and combinations thereof, and wherein said cell is negative for a marker selected from CD31, CD34, CD45, and combinations thereof.

Urine stem cells may be collected from any animal that produces urine, including humans. In some embodiments of the present invention, urine stem cells are collected from the urine of a mammal. For example, USC may be collected from the urine of a dog, cat, pig, cow, horse, monkey or human. In particular embodiments, urine stem cells are obtained from the urine of a human.

Urine stem cells may be collected from any portion of the urinary tract. In some embodiments, USC are collected from the upper urinary tract (UUT) (kidneys, ureter), e.g., via a catheter such as a nephrostomy catheter. In other embodiments, USC are collected from the lower urinary tract (bladder, urethra), via a catheter such as a urinary catheter.

In some embodiments, USC are collected from samples of fresh spontaneous urine, or drainage urine through a urethral catheter or from a bladder wash. Urine samples can be centrifuged at 1500 RPM for 5 minutes at 4° C., the supernatant aspirated, and cells washed with a suitable solution such as phosphate-buffered saline (PBS). The PBS may optionally contain serum such as 5% fetal bovine serum (FBS), and/or an antibiotic such as 1% penicillin-streptomycin to protect cells from injury and potential infection, respectively.

Further examples of methods and apparatuses for isolating cells from biological fluids may be found in, e.g., U.S. Pat. No. 5,912,116; U.S. Patent Application No. 20040087017; U.S. Patent Application No. 20020012953; and WO 2005/047529.

In some embodiments USC will double upon growing for 24-48 hours (e.g., every 31.3 hours), allowing them to be grown in large quantities. In further embodiments, USC do not induce tumor formation (as compared to embryonic stem cells), and in some embodiments USC do not require feeder cells for growth or differentiation.

### II. Scaffolds

“Scaffold” as used herein may include synthetic scaffolds such as polymer scaffolds and porous hydrogels; non-synthetic scaffolds such as pre-formed extracellular matrix layers, dead cell layers, and decellularized tissues; and any other type of pre-formed structure that aids in forming the physical structure of a cell-scaffold construct. Scaffolds may be made of natural bio-matrixes, such as spider silk, chitosan, and microspheres made from collagen, gelatin, fibrinogen, hyaluronic acid, and/or alginate; and/or synthetic materials, such as PGA37, PLGA40, and/or PLLA. In preferred embodiments disclosed herein, scaffolds are three-dimensional (3D) and comprise silk fibroin, which is an insoluble protein present in cocoon silk (e.g., of Bombyx mori).

In some embodiments, the scaffold is formed by electrospinning. Electrospinning, and electrospinning of silk fibroin, is known and described in, for example, U.S. Pat. No. 7,842,780 and US Publication No. 2005/0260706 to Kaplan et al. In some embodiments, the scaffold has fiber sizes of from 1, 3 or 5 micrometers, to 15, 20, or 30 micrometers in diameter.

In some embodiments, the scaffold has mixed-sized fibers (i.e., a mix of thicker and thinner fibers) in a range of from 1, 3 or 5 micrometers, to 15, 20, or 30 micrometers in diameter. Without wishing to be bound by theory, it is thought that with such a configuration the silk macro-fibers (thick silk fibers: from 15 to 30 micrometers in diameter (e.g., about 20 micrometers in diameter)) may act as pillars to strengthen the scaffolds, while microfibers (thin fibers: from 1 to 5 micrometers in diameter (e.g., about 3 micrometers in diameter)) form the network with bundles to link and/or aggregate cells. Thus, the mechanical properties of mixed sized fibers may provide improved scaffolds for 3D cell culture of the cells in some embodiments.

In some embodiments, electrospun silk fibroin fibers are crosslinked. See, e.g., Mu et al., Polymers (Basel) 2020 December; 12(12):2936. In some embodiments, silk fibers are crosslinked with an alcohol such as ethanol or methanol.

In some embodiments, the scaffold has pores with sizes of from 30, 35 or 40 micrometers, to 70, 75, 80 or 85 micrometers. In some embodiments, the scaffold has a porosity of 70, 75, 80, or 85% or greater.

### III. 3D Construct and Methods of Making

An in vitro 3D construct as taught herein may be made, for example, by: providing a 3D scaffold comprising silk fibroin and having a crosslinked porous matrix; seeding the 3D scaffold with stem cells and allowing the stem cells to adhere to the 3D scaffold; and growing the stem cells on the 3D construct for a time of from 2 or 4 weeks, to 6, 8 or 10 weeks, to thereby make the in vitro construct. In some embodiments, the 3D scaffold is formed by electrospinning a composition comprising the silk fibroin, and then crosslinking the silk fibroin.

In some embodiments, the stem cells adhered to form the 3D construct have stable mitochondrial DNA (mtDNA) when grown for 2 or 4 weeks, to 6, 8 or 10 weeks (e.g., at least 6 weeks in culture). “Stable mitochondrial DNA” as used herein refers to a substantially constant copy number or amount/content of mitochondrial DNA over a period of time or as measured at two or more different points in time, indicating that mitochondrial DNA is not undergoing depletion (which may be associated with mitochondrial dysfunction), or mitochondrial biogenesis, which may indicate replication. See Chiappini et al., Laboratory Investigation 84 (2004): 908-914. For example, mitochondrial DNA content of the cells may vary less than 20, 10, or 5% at 4 or 6 weeks of culture as compared to week 2 of culture. See Ploumi et al., FEBS J. 284 (2017): 183-195.

The stem cells may be grown in any suitable media. “Media” as used herein may be any natural or artificial growth media (typically an aqueous liquid) that sustains the cells used in carrying out the present invention. Examples include, but are not limited to, an essential media or minimal essential media (MEM), or variations thereof such as Eagle's minimal essential medium (EMEM) and Dulbecco's modified Eagle medium (DMEM), as well as blood, blood serum, blood plasma, lymph fluid, etc., including synthetic mimics thereof.

In some embodiments, USC are grown in keratinocyte serum-free medium (KSFM) and progenitor cell medium (1:1). See Zhang et al., J. Urol. 180 (2008) 2226-2233. For example, KSFM may be supplemented with one or more of 5 ng/ml epidermal growth factor, 50 ng/ml bovine pituitary extract, 30 ng/ml cholera toxin, 100 U/ml penicillin and 1 mg/ml streptomycin. Progenitor cell medium may contain ¾ Dulbecco's modified Eagle's medium, ¼ Hamm's F12, 10% fetal bovine serum (FBS), and one or more of 0.4 μg/ml hydrocortisone, 10⁻¹⁰ M cholera toxin, 5 ng/ml insulin, 1.8×10⁻⁴ M adenine, 5 μg/ml transferrin plus 2×10⁻⁹ M 3,39,5-triiodo-L-thyronine, 10 ng/ml epidermal growth factor (EGF), 10% penicillin and streptomycin.

In some embodiments, the in vitro construct is grown as a dynamic culture, in which the media is circulated or otherwise moving with respect to the scaffold. For example, the construct may be grown with dynamic culture in an orbital shaker (e.g., at about 40 RPM).

In some embodiments, the construct seeded with stem cells further comprises macrophages, endothelial cells and/or stromal cells (e.g., human primary macrophages, endothelial and/or stromal cells). In some embodiments, macrophages, endothelial cells and stromal cells are included with USC in a ratio of about 1:1:1:7, respectively (i.e., 1 macrophage:1 endothelial cell:1 stromal cell:7 USC).

### IV. Methods of Use for Toxicity Testing

As noted hereinabove and below, the in vitro constructs of the present invention are particularly useful in methods of toxicity testing, in which a long-term culture for chronic toxicity testing as well as large numbers of cells for serial analysis of gene and protein expression are desirable.

“Subjects” as used herein are, in general, human subjects, although aspects of the invention may be implemented with other animal subjects, particularly mammalian subjects (e.g., dogs, cats, horses, goats, sheep) for veterinary purposes. Subjects may be male or female and of any age.

“Assay,” as used herein, may be any procedure for testing or measuring the presence or activity of a substance of interest in a sample (e.g., an in vitro construct, cell aggregate, tissue, organ, organism, etc.).

The “substance of interest” may be, for example, a chemical (such as an environmental toxicant or industrial chemical, or chemical used in consumer products), a biochemical (such as a protein or hormone), or a drug (such as a small molecule drug, biologic, etc.). See, e.g., Varga, et al., Drug-induced mitochondrial dysfunction and cardiotoxicity. Am. J. Physiol. Heart Circ. Physiol. 2015, 309, H1453-467; Schnegelberger et al., Environmental toxicant-induced maladaptive mitochondrial changes: A potential unifying mechanism in fatty liver disease?Acta Pharm. Sin. B 2021, 11, 3756-3767; Attene-Ramos et al., Systematic Study of Mitochondrial Toxicity of Environmental Chemicals Using Quantitative High Throughput Screening. Chem. Res. Toxicol. 2013, 26, 1323-1332; Wills, The use of high-throughput screening techniques to evaluate mitochondrial toxicity. Toxicology 2017, 391, 34-41.

“Toxicity” as used herein may be any adverse effect of a substance of interest such as a drug on a living organism (subject) or portion thereof. The toxicity can be to individual cells, to a tissue, to an organ, or to an organ system. A measurement of toxicity is useful for determining the potential effects of the drugs on human or animal health, including drugs intended for long term administration to a patient, and/or the significance of drug or other chemical exposures in the environment.

In some embodiments, toxicity may be measured by providing the in vitro construct as taught herein; contacting the construct with a substance of interest for a period of time (e.g., from 2 or 4 weeks, to 6, 8 or 10 weeks); and detecting a biological response of the stem cells of the construct, wherein the biological response may indicate toxicity of the substance of interest. In some embodiments, biological response may include acute and/or chronic toxicity, including cytotoxicity and/or mitochondrial toxicity in organs or tissues, such as the liver, heart, brain/periphery nerve, skeletal muscle, blood cells, kidneys (i.e., hepatotoxicity, cardiotoxicity, neurotoxicity, myotoxicity, hematotoxicity or nephrotoxicity, respectively).

Cytotoxicity (apoptosis or cell death) may be measured by determining the number or percentage of cells that are damaged or do not survive during the period of time. Mitochondrial toxicity may be measured, for example, by determining the stability of mitochondrial DNA content, the reactive oxygen species (ROS) level, mitochondrial membrane potential, mitochondrial swelling, cytochrome c release, and Complex I-V activity in the cells, by measuring mitochondrial function such as the inhibition of oxidative phosphorylation complexes, etc. Nephrotoxicity may be measured by determining the cytotoxicity, renal cell marker expression, mitochondrial function, organic anion transport, and ultrastructure of kidney cells (e.g. USC).

Drugs that may be tested in the assays taught herein may include, but are not limited to, antiretroviral therapy (ART) drugs, anti-diabetic drugs (such as thiazolidinediones, fibrates, biguanides), cholesterol lowering drugs (such as statins), anti-depressants (such as SARIs), pain medications (such as NSAIDs), antibiotics (such as fluroquinolones, macrolide), and anti-cancer drugs (such as kinase inhibitors and anthracyclines) etc., including combinations thereof.

See also Ding et al., “Silk Fibers-Assisted 3D Culture of Human Urinary Stem Cells Suitable for Chronic Mitotoxicity Testing,” (preprint published Oct. 26, 2021); and Ding et al., “Silk fibers assisted long-term 3D culture of human primary urinary stem cells via inhibition of senescence-associated genes: Potential use in the assessment of chronic mitochondrial toxicity,” Materials Today Advances 15:100261 (published online June 2022).

The present invention is further described in the following non-limiting examples.

### EXAMPLES

Example 1: Development of Improved 3D Cell Culture System for in vitro Toxicity Testing. We evaluated a long-term culture of human primary urine stem cells (USC) seeded in 3D silk fiber matrix (3D USC-SFM) and tested chronic mitochondrial toxicity induced by Zalcitabine (ddC, a nucleoside reverse transcriptase inhibitor) as a test drug, compared to USC grown in spheroids. The numbers of USC remain steady in 3D spheroids for 4 weeks and 3D SFM for 6 weeks. However, the majority (95%) of USC survived in 3D SFM, while cell numbers significantly declined in 3D spheroids at 6 weeks. Highly porous SFM provides large-scale numbers of cells by increasing the yield of USC 125-fold/well, which enables the carrying of sufficient cells for multiple experiments with less labor and lower cost, compared to 3D spheroids. The levels of mtDNA content and mitochondrial superoxide dismutase2 (SOD2) as an oxidative stress biomarker and cell senescence genes (RB and P16, p21) of USC were all stably retained in 3D USC-SFM, while those were significantly increased in spheroids. mtDNA content and mitochondrial mass in both 3D culture models significantly decreased six weeks after treatment of ddC (0.2, 2, and 10 mM), compared to 0.1% DMSO control. Levels of complexes I, II, and III significantly decreased in 3D SFM-USC treated with ddC, compared to only complex I level which declined in spheroids. A dose- and time-dependent chronic MtT displayed in the 3D USC-SFM model, but not in spheroids.

Thus, 3D USC-SFM as a long-term 3D culture model of human primary USC provides a cost-effective and sensitive approach for the assessment of drug-induced chronic mitochondrial toxicity.

### Material and Methods

Silk fibroin was extracted from silk cocoons (TTSAM, China) according to the methods as previously reported (Ding et al. Mater. Sci. Eng. C Mater. Biol. Appl. 71 (2017) 222e230). Zalcitabine (ddC) is a well-known anti-HIV drug inducing MtT as a test drug in this study, which was provided from the NIH HIV reagent program (www.hivreagentprogram.org). Dimethyl sulfoxide (DMSO) is a known negative control for MtT and was purchased from Sigma (St. Louis, Mo.).

Human USC were cultured in combined media: keratinocyte serum-free medium (KSFM) and progenitor cell medium (1:1) as previously reported (Zhang et al. J. Urol. 180 (2008) 2226e2233). Briefly, KSFM was supplemented with 5 ng/ml epidermal growth factor, 50 ng/ml bovine pituitary extract, 30 ng/ml cholera toxin, 100 U/ml penicillin and 1 mg/ml streptomycin. Progenitor cell medium contained ¾ Dulbecco's modified Eagle's medium, ¼ Hamm's F12, 10% fetal bovine serum (FBS), 0.4 μg/ml hydrocortisone, 10¹⁰ M cholera toxin, 5 ng/ml insulin, 1.8×10⁻⁴ M adenine, 5 μg/ml transferrin plus 2×10⁻⁹ M 3,39,5-triiodo-L-thyronine, 10 ng/ml epidermal growth factor (EGF), 10% penicillin and streptomycin, were all purchased from Gibco (Thermo Fisher Scientific, Waltham, MA, USA). Acetone, ethanol, methanol, isopropanol, phosphate buffered saline (PBS) and all other reagents were used in this study. Demineralized water was used in all cases.

Silk fibroin (10%) electrospinning solution and random-structured matrix were collected using a wet process. Briefly, the sponge-like silk fiber matrixes (SFM) were assembled in a 100% ethanol (Warner Graham Company, USA) bath up to 45 min. After being fully cross-linked with ethanol and washed, the SFM samples were frozen with deionized water in a culture dish at dimeter 6 cm (Corning, NY). All SFM samples were lyophilized for 3 days.

Two sizes of SFM were made by Biopsy Dermal Punches (Painful Pleasures, USA) for different applications (Table 1): i) Small size SFM (s-SFM, 4 mm at diameter and 0.2 mm in thickness) fitted into a 96-well plate with ultralow attachment (ULA) U bottom (Corning, NY), was used for measuring cell growth curves, live/dead assays and immunofluorescence for SOD2; and ii) large size SFM (1-SFM, 8mmat diamante and 1 mm) fitted to 12-well or 6-well ULA plate (Corning, NY) was used for the evaluation of mitochondrial function (complex IeV) by Western blot and mitochondrial DNA copy number by q-PCR that requires large numbers of cells.

TABLE 1
3D culture systems of USC used in this study

| | 3D USC-SFM | | |
|---|---|---|---|
| | Large size SFM | Small size SFM | 3D USC |
| | (l-SFM) | (s-SFM) | spheroids |
| Cell no./well | 5 × 10⁵/Ø8 mm/well, 1 mm | 4 × 10³/Ø4 mm/well, 0.2 mm | 4 × 10³/well in |
| | thickness in ULA 12-well | thickness in ULA 96-well | ULA |
| | plate | plate | 96-well plate |
| Cell culture | 6 weeks | 6 weeks | 4 weeks |
| time periods | | | |
| Suitable for | Complex I-V and SOD₂ | Morphology assessed | Active mitochondria |
| toxicological | assessed by Western-blot | by phrase contrast, | assessed by Mito-Tracker |
| parameters | and immunostaining | Cell growth curve or cell | green with |
| | mt-DNA content, cell | viability by cck8 and | immunostaining |
| | Senescence-related genes | live/dead kits | Cellular respiration and |
| | by q-PCR | Ultrastructure by SEM | lactate release assessed |
| | | | by seahorse technology |

Abbreviations: ULA, ultralow attachment; cck8, Cell Counting Kit 8; mt-DNA, mitochondrial deoxyribonucleic acid; q-PCR, real-time polymerase chain reaction; FCM-flow cytometry; Immunostaining, immune-fluorescent staining; SEM, scanning electron microscope; MtT, mitochondrial toxicity.

Human urine samples were collected from 12 healthy male donors aged from 17 to 65 years. Cell pellets were washed with PBS following urine samples being centrifuged. The cells were plated in culture plates with USC medium. USC were cultured at 37° C. in a humidified atmosphere of 5% CO₂. Cells at passage (p) 3 were used for all the groups and tests. To assess cell morphology, proliferation and live/dead, USC were seeded into 96-well plates in three culture conditions: i) 2D culture (4×10³ cells/well); ii) 3D sphere in 96-well plates with ULA (4×10³ cells/well); iii) 3D s-SFM in 96-well plates with ULA (4×10³ cells/s-SFM/well), respectively. To generate larger numbers of cells for Western-blot analysis, USC were cultured either in 1-SFM for 3D USC-SFM in 12-well or 6-well ULA plates (5×10⁵ cells/1-SFM/well) or in Micro-molds (Microtissues 3D Petri Dish (Sigma, USA)) for 3D spheres with 88 wells. Culture media were changed every other day.

Cell viability of USC within spheroids and SFM was examined using a live/dead assay (Thermo Fisher) at week 1, 2, 4, 6 and 8. These time points are based on slow cell growth rates as the population doubling times ranged from 1 to 4 weeks in 3D spheroids depending on cell types (Ding et al. Pharmaceutics 14 (2022) 1042). Calcein AM and EthD-1 were diluted by PBS into 2 mM and 4 mM working solutions, Cell construct samples were washed with PBS, and incubated in the working solution for 30 min in room temperature (RT). After being washed with PBS following staining, USC within 3D culture samples were observed under confocal microscope (Leica TCS-LSI, Leica Biosystems Inc. Buffalo Grove, IL) or an Olympus IX-70 fluorescence microscope.

Cell proliferation or viability was measured at different time points after USC were seeded on 2D, spheroids and 3D-SFM in 96-well plates, respectively, assessed by Cell Counting Kit-8 (CCK-8 assay, Dojindo, Japan), according to manufacturer's instructions. The absorbance at 450 nmwas measured using microplate reader (MultiSkan FC, Thermo, USA).

To evaluate the maximum amount of cell numbers capable of being cultured on 3D USC-SFM, different cell numbers (10⁶, 10⁵, 10⁴, and 4×10³ cells/well) in 220 ml culture medium were seeded on s-SFM in ULA 12-well plates for 6 weeks and assessed by CCK-8 as above. To allow cells to efficiently attach to the SFM scaffolds, USC seeded on s-SFM were incubated for 2 h and then extra 200 ml culture medium was added. USC-SFM were transferred from 96-well to 12-well plate 12 h after initial seeding with enough medium the next day.

A scanning electron microscope (SEM) was used to evaluate the surface morphology of spheroids and USC-SFM. Both types of 3D culture samples were fixed in 2.5% glutaraldehyde and dehydrated using a Leica EM CPD300 Critical Point Dryer (Leica Microsystems GmbH, Wetzlar, Germany), then mounted and sputter-coated with gold sputtering. The cell samples were examined under FlexSEM 1000 Scanning Electron Microscopy (Hitachi Medical Systems America Inc., Twinsburg, OH, USA) at an accelerating voltage of 10 kV and working distance of 6 mm.

To determine the dose effect of ddC on cell viability, mitochondrial function and oxidative stress of USC in both 3D cultures, ddC was added to spheroids and USC-SFM at different doses: 0.2, 2 and 10 μM in the culture medium every 2 days, 3 replicates per concentration. To evaluate the time-dependent effect of ddC on USC in 3D cultures, spheroids and USC-SFM samples were assayed 2-6 weeks after administering ddC, 3 replicates per time point. DMSO (0.1%) was used as a control.

To assess mitochondrial superoxide dismutase 2 (SOD2) levels, both spheroids and USC-SFM samples were fixed with 4% PFA for 30 min, permeabilized by 0.2% Triton-X 100, and blocked by DAKO protein block. The cell samples were incubated in primary antibodies anti-human SOD2 (Cell Signaling Technology, USA, diluted 1:50, Danvers, MA) overnight at 4° C. Secondary antibody (Goat anti-Mouse, Alexa Fluor™ 647, Thermo Fisher Scientific, USA, diluted 1:200) was then applied for 2 h at RT. Cell nuclei were stained by DAPI and then observed by an Olympus FV10i® confocal laser scanning microscope.

To measure the levels of cell senescence genes (p13, 21 and Rb) (Kang et al. Science 349 (2015) aaa5612) and mitochondrial DNA (mtDNA) content, mRNA expression of spheroid (n ¼ 3) and 3D USC-SFM (n ¼ 3) samples were assessed using BioRad CFX connect Real-time PCR Detection System (Rooney et al. Methods Mol. Biol. 1241 (2015) 23e38). Genomic DNA was extracted from the cells using a DNeasy Blood & Tissue Kit (Qiagen, Valencia, Cat. No 69504) according to the manufacturer's protocol. The Q-PCR recipe was a mix with SYBR Green SuperMix (ThermoFisher, USA, Cat. No 4367659) using both the mitochondrial and the nuclear primers, and this temperature cycling was used: initial denaturing at 50° C. for 2 min, 95° C. for 15 min, followed by 40 cycles of denaturing at 95° C. for 30 s, annealing at 60° C. for 1 s and extension at 95° C. for 15 s, 60° C. for 1 min, annealing at 95° C. for 15 s, 60° C. for 1 min and dissociating at 95° C. for 15 s.

To determine mRNA expression of ddC-treated USC in 3D cultures, the mRNA was extracted by RNeasy Mini Kit (Qiagen, Valencia, Cat. No 74104) and reverse transcribed to cDNA by High-Capacity cDNA Reverse Transcription Kit (Thermo Fisher, USA, Cat. No 4368814). The other reagents and the primers were the same as Real time-PCR for mtDNA. This PCR temperature cycling was used: initial denaturing at 50° C. for 2 min, 95° C. for 10 min. The rest of the process was the same as the Real time-PCR for mtDNA. The reference is normalized by the geometric mean of GAPDH, POLR2A and PGK1.

To generate sufficient cell numbers for immunoblot analysis, USC (5×10⁵ cells) within spheroids and 1-SFM were loaded in 81-well molds made by Micro-Tissues 3D Petri Dish (Sigma, USA). USC in 2D culture were seeded onto a 6-well plate at a density of 5×10⁵ cells/well as control. After a wash of the cell samples with PBS, the USC were harvested and incubated for 30 min in the presence of 500 ml of lysis buffer (Pierce, Rockford, IL) with 1% protease/phosphatase inhibitor cocktail (Cell Signaling Technology, Danvers, MA), vortexing every 5 min during incubating. The lysate was clarified by centrifugation, and protein concentrations were tested by Pierce™ BCA Protein Assay Kit. Following separation in 15% SDS-PAGE gels, the proteins were transferred onto a PVDF membrane (ThermoFisher) by a Bio-Rad Trans-Blot SD Semi-Dry Transfer Cell under 12 V for 1 h.

Individual activity of oxidative phosphorylation complexes I, II, II IV, V94 and mitochondrial SOD2 in USC before or after treatment with ddC at three doses were assessed. The membrane was blocked for 30 min in PBS-0.1% Tween 20 (PBST) containing 5% bovine serum albumin (BSA), washed with PBST, and incubated with the primary antibodies for 2 h or 4° C. overnight, diluted by PBST containing 5% BSA. After extensive washing with PBST, the membrane was incubated in the secondary antibodies correspondingly for 1 h at room temperature. The washed membrane was treated with an Immobilon ECL Ultra Western HRP Substrate (Millipore Sigma) and analyzed with a Fujifilm LAS-3000 Luminescent Image Analyzer system.

Descriptive statistics are presented as mean±standard deviation (SD), GraphPad Prism software version 9.0. All data shown are derived from experiments that were independently repeated at least three times. For any experiment with multiple treatments, such as different doses, one-way ANOVA with Dunnett's multiple comparisons to control group (DMSO) or multiple unpaired t tests were used. For any experiment with two groups, such as different time points, student's t-test was used to make comparisons. Bonferroni's multiple comparisons were applied whenever appropriate. For all analyses, overall statistical significance was set at a=0.05.

### Results

### Fabrication of 3D Silk Fiber Matrix

The diameter of silk fibers, porosity, and pore size of the SFM were varied and characterized, leading to direct implications for cellular functionality and cell behavior in vitro. Both s-SFM and 1-SFM were fabricated (Table 1). The 3D SFMs at different sizes retained the entire porous structure without degradation in culture medium for more than 10 weeks. SFM was not affected by lysis buffer during protein or DNA/RNA extraction processes. In addition, SFM alone did not affect protein and gene expression assayed by Western blot and q-PCR.

### Cell Growth and Viability of USC

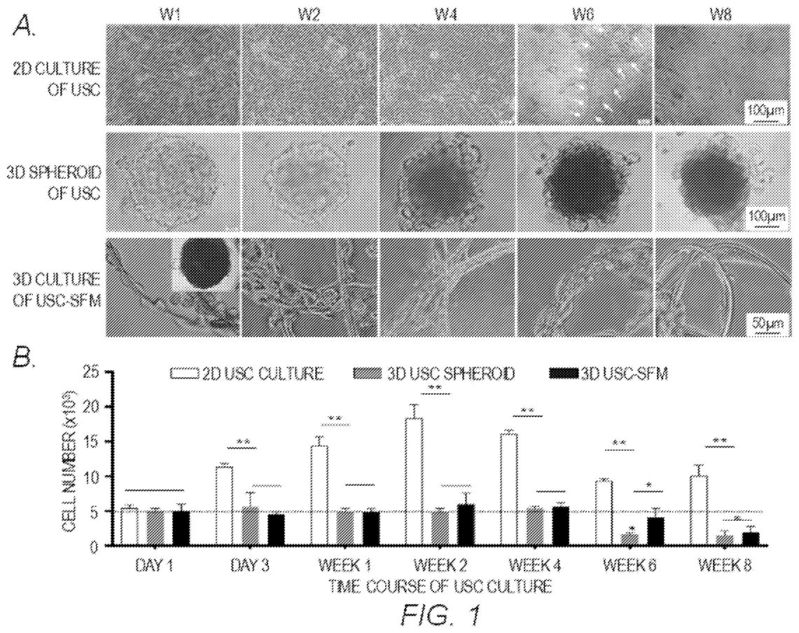

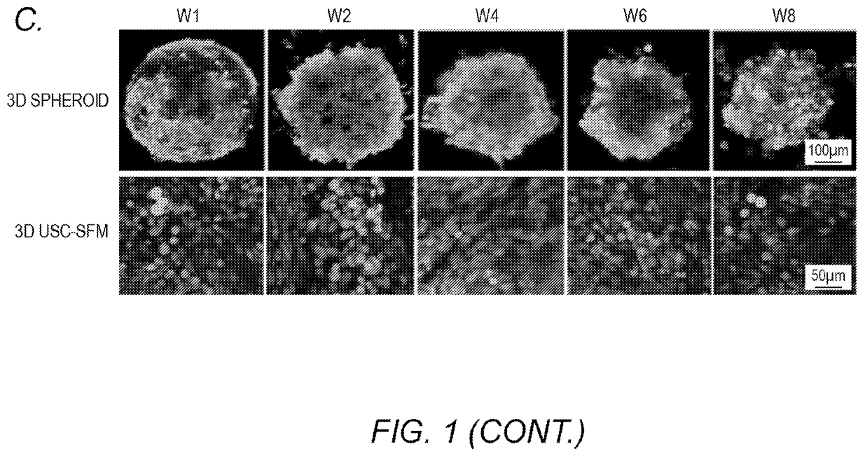

In 2D culture, USC constantly proliferated and displayed overconfluence with the number of cells reaching the peak at week 4 and starting to decline at weeks 6 and 8 (FIG. 1, Panels A and B). Cells formed “hills” with multiple layers on some areas (white arrows) and “valleys” with a single layer of cells on other areas at 6 weeks. This was due to aging USC that detached while the remaining USC with self-renewing properties started to proliferate and covered a “valley” region. Finally, some USC in 2D culture detached, and some cells at large size were still left on the culture dishes 8 weeks after initial plating.

The sizes of 3D spheroids remained similar during the 8-week culture; however, cells at the outer layers became larger in size, and the bodies of spheroids appeared semi-transparent at 2 weeks but became darker at week 6 and 8, indicating that unhealthy cells existed in the center of spheroids with time. The solid cell spheroids lacked channels or spaces from the outer layers to the center of 3D spheroid.

In 3D SFM, USC attached, grew along silk fibers, and aggregated at the connecting points of silk fibers in the 3D constructive matrix, and more spaces among cells opened during 8-week culture to allow the culture medium flow passing through (FIG. 1, Panel A). When cell growth curves and live/dead analysis were performed in 3D s-SFM, the number of USC was stable in s-SFM for 6 weeks, then declined at week 8; whereas the number of cells remained constant in 3D spheroids at week 4 but significantly decreased at week 6 (p<0.01, FIG. 1, Panel B). In addition, live/dead analysis showed an intense green fluorescence signal for live cells and a faint red fluorescence signal for dead cells, indicating the majority cells remained viable in the 3D silk fiber network after 2, 4, 6 and 8 weeks of incubation. Although the number of cells decreased, higher viability was observed in 3D SFM at week 8. In contrast, 3D USC spheroids displayed an intense red fluorescence at the center at week 6 (FIG. 1, Panel C), indicating substantial cell death and matching the number of cells in spheroids decreasing at week 6. Thus, the ultimate timing to test late mitochondrial function in 3D USC-SFM was found to be week 6 while the proper timing for testing drug induced MtT in 3D spheroids was at week 4.

### Mitochondrial DNA Content and Ultrastructure of USC

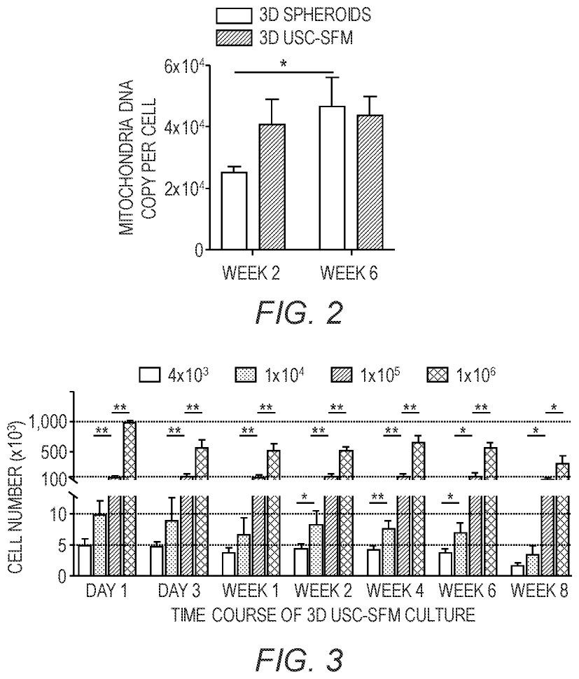

USC did not proliferate in 3D SFM and spheroids (FIG. 1, Panel B), and mitochondrial DNA (mtDNA) copy numbers were similar in 3D USC-SFM between 2 and 6 weeks after culture (FIG. 2), which is preferred for mitochondrial function testing because the copy number of mitochondrial retained stable without replicating. In contrast, levels of mtDNA content in 3D spheroids significantly increased from weeks 2-4. Mitochondrial biogenesis is the cellular process that produces new mitochondria, which effectively causes an increase in mitochondrial mass (Ploumi et al. FEBS J. 284 (2017) 183e195). Thus, mitochondrial biogenesis of 3D USC-SFM remained stable as compared to spheroids in 6-week culture. These data indicate that mitochondrial DNA copy number remains more consistent in 3D USC-FSM than that in spheroids, which makes USC-FSM more suitable for MtT testing.

Ultrastructurally, the apical membrane facing the tubular lumen of normal renal proximal tubule epithelial cells (RPTEC) is folded and covered by brush border (microvilli) that increases transport area as an absorptive function characteristic (Weinbaum et al. Am. J. Physiol. Ren. Physiol. 299 (2010) F1220eF1236). Microvilli appeared on the surface of USC in 3D SFM and spheroids during 4-week culture (not shown), indicating that 3D cultures of USC possess certain reabsorption properties of RPTEC.

Ample Numbers of USC Aggregated within SFM

To determine the maximal cell numbers of USC loaded in 3D SFM, we used different cell numbers (10⁶, 10⁵, 10⁴, and 4×10³ cells/s-SFM sample/well) in 220 ml culture medium in 12-well ULA plates for 8 weeks, assessed by CCK-8. When USC at 10⁶ cells/well are loaded into s-SFM, only ½ of USC (i.e., 5×10⁵ cells) were retained 24 h after seeding and the half of cells were washed away following medium changes, indicating that the maximal number of cells carried in s-SFM are 5×10⁵ cells/s-FSM (O at 4×0.2 mm³). The number of USC in s-SFM at 4×10³, 1×10⁴, 1×10⁵, and 5×10⁵ cells/well were stably retained without significant cell proliferation during 6-week culture, but significantly decreased at week 8 (p<0.05) (FIG. 3).

Although s-SFM can carry a maximum number of cells at 5×10⁵ cells with high cell viability for 8 weeks, the medium must be changed two times a day to maintain cell viability when cultured in 96-well plates. To perform a better comparison of the cell growth curve and viability, we seeded the same cell density at 4×10³ USC in 2D culture, spheroids and 3D SFM, respectively. The CCK-8 test provided a reliable assay to test cell viability for 2D cultures or for small size 3D cultures or spheroids.

USC aggregated and attached at the silk fiber and the network connecting points to form 3D cell constructs with a large number of cells (5×10⁵ cells/well) in 1-SFM, which is about 125-fold higher than USC grown as 3D spheroids in 96-well plates (4×10³ cell/well). Serial MtT assessments often require up to 5×10⁶ cells/sample (Brown et al. mBio 6 (2015) e01741ee01815), and 3D 1-SFM in a 12-well plate provides enough cells (6×10⁶/plate) to meet these large cell number requirements.

### Mitochondrial SOD2 Expression in USC

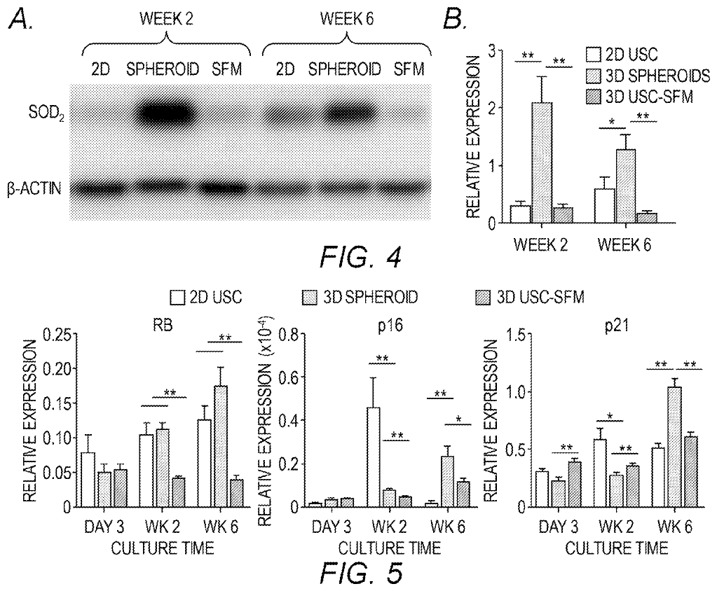

Western blot analysis showed that protein levels of mitochondrial SOD2 (mt-SOD2) in 3D USC-SFM and 2D culture remained low 2 and 6 weeks after culture, while protein levels of SOD2 significantly increased in 3D USC spheroids (FIG. 4). Dual immunofluorescence staining also demonstrated that the expression levels of SOD2 in 3D USC-SFM and 2D culture were significantly lower than those in spheroids. Because of a mitochondrial antioxidant role of SOD2, the increased expression of SOD2 indicates that USC in spheroids were under more oxidative stress than in 2D culture or 3D USC-SFM.

### Senescence Gene Expression in USC

The expression of senescence-related genes (p13, 21 and Rb) of USC in 3D USC-SFM was significantly lower than those in 3D spheroids (p<0.05) (FIG. 5). This indicates that silk-fiber networks provide the appropriate microenvironment for USC growth by preventing the expression of senescence-related genes in 3D structures during long-term culture. In addition, lower expression of senescence-related genes was found in 2D culture. This might be due to the occurrence that only USC with self-renewal potential remain on the 2D cell dish and continue to overgrow or that aging cells were detached and washed away during 6-week culture. In addition, cells in the 2D culture have easier access to the medium for metabolites and nutrient exchange, potentially leading to increased proliferation.

ddC-Induced Chronic Cytotoxicity Assessment

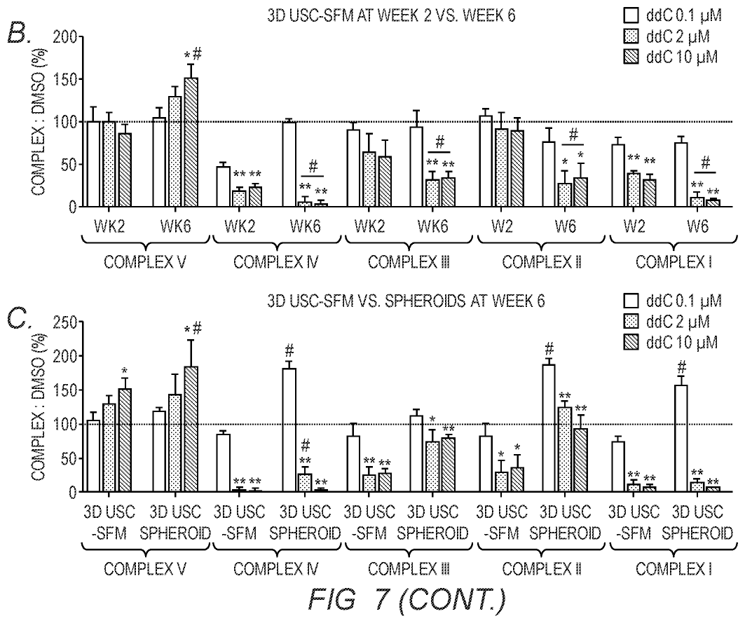

The antiretroviral drug ddC significantly affected cell viability and growth of USC in 2D culture. Cells readily detached with few cells remaining on the dishes 2 weeks after ddC treatment. In contrast, ddC did not significantly affect cell survival and growth at 3 days, or 1, 2, 4, and 6 weeks after culture in 3D USC-SFM (FIG. 7, Panel A).

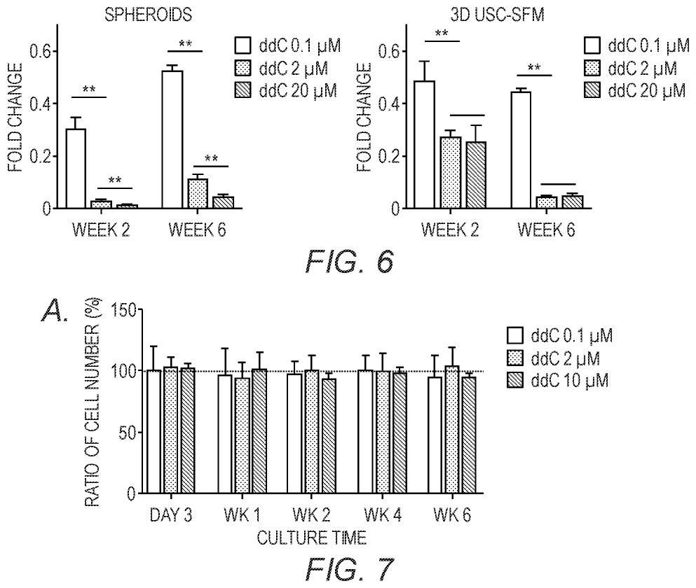

Levels of mtDNA content significantly decreased in 3D spheroid of USC and 3D USC-SFM 2 and 6 weeks after being treated with ddC at three different doses (0.2, 2, or 20 μM), compared to cells treated with DMSO standardized as 100% (FIG. 6).

Levels of Oxidative Phosphorylation Complexes I-V Decreased after ddC Treatment

MtT assessments showed that ddC induced significant impairment of mitochondrial function by inhibiting oxidative phosphorylation complex I-IV in 3D culture models, which featured as: i) in FIG. 7, Panels B and C, it shows that the levels of complex I, and IV significantly decreased 2 weeks after being treated with ddC at middle and high doses (2 and 10 μM), compared to ddC at the low dose (0.1 μM); the levels of complex I, II, III and IV significantly declined 6 weeks after treated with middle and high dose ddC, compared to those in ddC at low dose. These data indicate that the dose-dependent MtT of ddC can be detected in 3D USC-SFM during 6 weeks of culture; ii) complex I-IV levels in 3D USC-SFM significantly decreased in response to ddC at 2 and 10 μM at 6 weeks compared to 2 weeks, indicating a time-dependent MtT of ddC developed in 3D USC-SFM model; iii) the levels of complexes II, III and IV in 3D USC-SFM significantly decreased by ddC, compared to complex levels in spheroids. In addition, levels of complexes I, II and IV significantly increased after being treated with low dose ddC in 3D spheroids, which did not occur in 3D USC-SFM. The data indicate that the 3D USC-SFM model was more sensitive and reliable to detect drug-induced MtT, as compared to 3D spheroids.

Thus, the data indicate that ddC induced mild cytotoxicity but a significant dose- and time-dependent mitochondrial toxicity as represented by decreasing levels of complexes I-IV in USC in both spheroids and 3D SFM, but that 3D USC-SFM is more sensitive in detecting oxidative phosphorylation complex proteins than 3D spheroids.

The present study demonstrated that a long-term in vitro 3D culture system of human primary USC can predict MtT that 2D culture cannot achieve. In vitro 3D culture systems are superior to 2D in long-term culture and more accurate in testing chronic mitochondrial toxicity, with neither cell proliferation nor mitochondrial replication occurring in the 3D USC cultures. 3D spheroid assays can be used in the measurement of the parameters requiring immune-fluorescence staining, and the evaluation of cellular respiration and lactate release with Seahorse technology. In addition, 3D USC-SFM can carry an ample number of cells for 6 weeks, are more sensitive and reliable in testing MtT, and are more physiologically relevant than 3D spheroids. Thus, 3D USC culture systems provide cost-effective and sensitive assays with less labor and reduced cost to test toxicant or drug induced chronic MtT via a series of experiments, compared to traditional 2D cultures.

We have developed novel in vitro 3D models with unique cell sources and natural silk fiber biomaterials which yield large scale production of long-term cultured human primary USC for a series of mitochondrial functional analysis. In vitro 3D long-term culture platforms of USC provide new opportunities in testing chronic MtT for new drug development and personalized toxicology. In vitro long-term 3D cultures developed in this study yield reproducible dose- and time-dependent chronic MtT, which is not possible in existing 2D cultures. Particularly, these in vitro 3D USC assays can be used for testing nephrotoxicity as USC are renal progenitor cells.

Because of the complexity of the in vivo system where multiple tissues are affected by drug toxicity, in ongoing studies, we are modifying 3D models by loading multiple cell types and ECM supporting specific tissue function to test various drugs. In the future, such 3D culture assays designed with USC from the patients with HIV or healthy individuals with pre-exposure prophylaxis could help determine whether chronic MtT is induced after long-term of ART. In addition, HIV itself could contribute to mitochondrial dysfunction. HIV infection-induced mitochondrial dysfunction and premature T cell aging will also be studied. Thus, we can better understand and assess the ART-mediated MtT in the setting of HIV infection for the individual taking ART for life-long treatment.

Not only can 3D assays of human primary stem cells limit the number of unsafe drugs, but they also promise to allow a whole generation of new drugs to prosper. It may also allow for a personalized in vitro system to predict individual susceptibility and assess chronic MtT of new drugs, particularly for lifelong anti-HIV drugs.

### Example 2: Further Development of Silk Fiber Matrix Properties

We evaluated how fiber size, porosity, and pore size of the silk fiber matrix (SFM) affects the long-term 3D cultures of USC for chronic MtT assessment. We seeded USC within an electro-spun SFM having different physical properties. USC (p4, 5×10⁵/SFM) were seeded on SFM (10 mm at 0, and 4 mm at thickness) with different diameters of silk fibers (nano-size [<3 μm], micro-size [<20 μm], and mixed-size matrix [3-20 μm]), pore sizes (10-40 μm, 41-80 μm, 81-120 μm) and porosities (50%, 65%, and 80%) of SFM.

We found that half a million cells can be grown well with 95% cell viability in 3D SFM with mixed-size fibers (3-20 μm), a pore size of 41-80 μm, and high porosity (>80%) for 6 weeks, which provides large numbers of cells for multiple mitochondrial function tests from one sample. In contrast, we found that cells seeded only onto the surfaces of SFM when nano-sized fibers (<3 μm), small pore sizes (10-40 μm) or low porosity (<50%) were used; and most cells sink to the bottom of the SFM with large-sized fibers (>20 μm), or large pore sizes (81-120 μm).

Thus, we found that USC are ideally grown on SFM with mixed-size fibers (e.g., 3-20 μm), a pore size of 41-80 μm, and high porosity (>80%).

### Example 3: Further Development of USC-SFM Culture Conditions

In an effort to better mimic in vivo tissue, human macrophages, endothelial cells, and stromal cells are added to 3D USC cultures.

To determine if a porous SFM in 3D dynamic culture (e.g., about 40 revolutions per minute (RPM) with an orbital shaker) improves longer cell survival, cell viability and mitochondrial function is measured in 3D USC-SFM up to 8 weeks, and compared to static cultures (and optionally cultures at lower speeds, e.g., 10 and 20 RPM).

To determine if porous SFM retains stemness and prevents cell senescence, telomerase activity and cell senescence-related proteins (p21, P53, p16, and Rb) are measured. Cell-cell interaction and cell-matrix adhesion are monitored.

Dynamic 3D USC cultures together with multiple cell types in porous SFM may provide a long-term culture model for toxicity testing by optimizing cell-cell and cell-matrix interactions and mitochondrial function, and by maintaining stemness while inhibiting senescence pathways.

### Example 4: Toxicity Testing of Drugs with USC-SFM

MtT profiles of six first-line drugs with unknown MtT: dolutegravir, bictegravir, raltegravir, elvitegravir, islatravir, and darunavir, are assessed and compared with nucleoside reverse transcriptase inhibitors (known MtT: DDC, D4T, DDI, and AZT; minimal MtT: FTC, and TAF). MtT is also tested in USC clinical samples from individuals on pre-exposure prophylaxis (Pr-EP) ART therapy. MtT is assessed by mitochondrial DNA content, Complex I-V, total ATP, C-caspase 3, and ROS/ribonucleotides for up to 6 weeks. Inhibition of Pol-γ48-50 and of ribonucleotide and deoxyribonucleotide pools is also quantified to examine the MtT mechanisms.

MtT and nephrotoxicity are determined for four anticancer drugs and two antibiotics with known toxicities, compared to drugs with minimal toxicity in 3D USC-SFM cultures. Long-term 3D cultures may more accurately predict chronic toxicities of ART, anticancer drugs, and antibiotics, compared to existing culture models (i.e., HepG2, microglia, adipocytes, and renal cells).

### Example 5: Correlation of Drug-Induced Toxicities Between the In Vitro USC-SFM Model and an In Vivo Model

MtT and nephrotoxicity are measured for subcutaneously implanted USC-SFM xenografts in male and female mice and compared to those in 3D cultures, and key organs including the kidneys in response to drugs listed in Example 4 at three doses after 6 weeks. 3D cultures of USC-SFM may represent a predictive in vitro model for in vivo drug-induced toxicities.

The foregoing is illustrative of the present invention, and is not to be construed as limiting thereof. The invention is defined by the following claims, with equivalents of the claimed to be included therein.

1. An in vitro construct comprising:
a three-dimensional (3D) scaffold comprising silk fibroin and having a crosslinked porous matrix; and
stem cells adherent to the 3D scaffold,
wherein the stem cells adherent to the 3D scaffold maintain stable mitochondrial DNA for at least 6 weeks in culture.

2. The construct of claim 1, wherein the stem cells are urine stem cells.

3. The construct of claim 1, wherein the stem cells are autologous to a patient in need of, or a candidate for, long term treatment with a drug.

4. The construct of claim 1, wherein the 3D scaffold comprises electrospun silk fibroin.

5. The construct of claim 4, wherein said electrospun silk fibroin comprises mixed-sized fibers from 1 to 30 micrometers in diameter.

6. The construct of claim 1, wherein the 3D scaffold has pores with sizes of from 40 to 80 micrometers.

7. The construct of claim 1, wherein the 3D scaffold has a porosity of 80% or greater.

8. The construct of claim 1, wherein the in vitro construct is grown as a dynamic culture.

9. The construct of claim 1, wherein the construct further comprises macrophages, endothelial cells and/or stromal cells.

10. A method of making an in vitro construct useful for toxicity testing, comprising:
providing a 3D scaffold comprising silk fibroin and having a crosslinked porous matrix;
seeding the 3D scaffold with stem cells;
allowing the stem cells to adhere to the 3D scaffold; and
growing the stem cells on the 3D construct for a time of from 2 or 4 weeks, to 6, 8 or 10 weeks,
to thereby make the in vitro construct.

11. A method of making the in vitro construct of claim 1, comprising:
providing the 3D scaffold comprising silk fibroin and having a crosslinked porous matrix;
seeding the 3D scaffold with the stem cells;
allowing the stem cells to adhere to the 3D scaffold; and
growing the stem cells on the 3D construct for a time of from 2 or 4 weeks, to 6, 8 or 10 weeks,
to thereby make the in vitro construct.

12. The method of claim 10, wherein the providing step is carried out by electrospinning a composition comprising the silk fibroin and then crosslinking the silk fibroin.

13. A method of performing toxicity testing, comprising:
providing the in vitro construct of claim 1;
contacting the construct with a substance of interest for a period of from 2 to 6 weeks; and
detecting a biological response of the stem cells, wherein said biological response indicates toxicity of the substance of interest.

14. The method of claim 13, wherein the biological response of the stem cells indicates cytotoxicity or mitochondrial toxicity in an organ or tissue.

15. The method of claim 14, wherein the organ or tissue comprises liver, heart, brain/periphery nerve, skeletal muscle, blood cells, or kidneys.

16. The method of claim 13, wherein the substance of interest is a drug.

17. The method of claim 16, wherein the drug is an antiretroviral therapy (ART) drug.

18. The method of claim 16, wherein the drug is selected from the group consisting of: anti-diabetic drugs, cholesterol lowering drugs, anti-depressants, pain medications, antibiotics, and anti-cancer drugs.

19. The construct of claim 1, wherein the construct further comprises human primary macrophages, endothelial cells, stromal cells, and USC provided at a ratio of 1:1:1:7, respectively.
